(19) United States
(12) Patent Application Publication
(10) Pub. No.: US 2018/0078163 A1
(43) Pub. Date: Mar. 22, 2018

(54) Biometric Reading Apparatus, System and Method of Use Thereof

(71) Applicant: Gregory P. Welch, Dallas, TX (US)

(72) Inventor: Gregory P. Welch, Dallas, TX (US)

(21) Appl. No.: 15/654,853

(22) Filed: Jul. 20, 2017

Related U.S. Application Data
(60) Provisional application No. 62/396,458, filed on Sep. 19, 2016.

(51) Int. Cl. A61B 5/0404; A61B 5/0452; A61B 5/1455; A61B 5/00; A61B 5/01; A61B 5/0245

(52) U.S. Cl. CPC A61B 5/0404; A61B 5/0452; A61B 5/14551; A61B 5/00; A61B 5/0002; A61B 5/01; A61B 5/0245; A61B 5/6802

(57) ABSTRACT

Biometric sensor devices, systems and methods for obtaining biometric data and displaying biometric information based on the data on the sensor devices. The systems allow for additional biometric data collection and/or analysis through wireless communication capabilities. The devices and systems provide real-time information to the medical professional working on a patient. The information may be further based upon medical guidelines associated with the performed medical activity.

### CROSS-REFERENCE TO RELATED APPLICATION

This application claims the benefit of priority from U.S. Patent Application No. 62/396,458 filed on Sep. 19, 2016, which is incorporated herein by reference.

### FIELD OF THE DISCLOSURE

The invention relates generally to the field of biometric sensing devices and systems.

### BACKGROUND

In healthcare, patient biometric information is used to assist medical professionals in evaluating, diagnosing and caring for a patient. Often medical practice steps are influenced by specific biometric data. Wearable sensor devices are sometimes used to collect biometric information. U.S. Patent Publication No. 2014/0275932 titled “Disposable Biometric Patch Device” is an example of a wearable sensor. The sensor detects data and transmits the data wirelessly to a remote device.

Acquiring physiological data for newborn infants presents a number of challenges. The information needs to be as accurate as possible to ensure the proper procedures are being followed for the specific situation. In addition, the information needs to be obtained as quickly as possible to provide the best possible outcome for the baby and to ensure the procedures follow guidelines which are often based upon the time from birth.

Medical guidelines for neonatal resuscitation immediately following birth are tied to the heartrate of the baby. See American Heart Association, Web-based Integrated Guidelines for Cardiopulmonary Resuscitation and Emergency Cardiovascular Care—Part 13: Neonatal Resuscitation, p. 6 (2015) (hereinafter “Neonatal Resuscitation Guidelines”). For example, the guidelines teach specific actions for when the heartrate is (1) above 100 beats/min, (2) between 60 and 100 beats/min and (3) below 60 beats/min. Notably, certain steps in the guidelines should be occurring within the first minute of the baby's life. In addition, these guidelines call for the use of a pulse oximeter to evaluate oxygenation at specific times after birth. This guideline illustrates the anticipated oxygenation levels based upon the time from birth with expectations from 1 minute old.

Medical professionals have historically relied upon (1) intermittently auscultating the precordial pulse and/or (2) palpating the infant's umbilical cord pulse. In addition, some had attempted to acquire a heartrate using a pulse oximeter. The 2015 guidelines were updated to recommend using a 3-lead ECG (electrocardiogram) to acquire a heartrate because clinical assessment using auscultation and pulse oximetry was unreliable and inaccurate. See Neonatal Resuscitation Guidelines p. 3. In addition, “pulse oximetry more often displayed a lower rate in the first 2 minutes of life, often at levels that suggest the need for intervention.” See Neonatal Resuscitation Guidelines p. 3. While the 3-lead ECG is recommended, application of a 3-lead ECG to a newborn takes time and often requires multiple wires connecting the leads on the child to a monitor located in the room.

Certain patents have presented devices that reduce or eliminate wiring for newborn ECG devices, such as U.S. Pat. Nos. 6,453,186 and 8,369,924. U.S. Pat. No. 6,453,186 is titled “Electrocardiogram Electrode Patch” and discloses a patch including a plurality of electrodes for attachment to an infant that includes a common connector to allow a single connection from a monitor in the room to each of the electrodes within the patch. Similarly, U.S. Pat. No. 8,369,924 is titled “ECG Leads System for Newborn ECG Screening” and discloses a chest strip ECG system having retractable limb leads for connecting to electrode leads to the infant's arms and legs. The ECG strip also may include a transceiver to transmit ECG signals to a receiver attached to an ECG machine located nearby.

Other patents and publications teach umbilical cord sensor systems for infant monitoring. For example, U.S. Patent Publication No. 2007/0276273 is titled “Periumbilical Infant ECG Sensor and Monitoring System” and discloses a combined ECG sensor and pulse oximetry sensor that attaches to the umbilical cord of the infant. The sensors are wired to the umbilical cord clamp then transferred to a monitor located nearby. Another example is U.S. Pat. No. 7,875,037 titled “Infant Umbilical Cord Cardiac Monitoring System and Method” which describes a monitoring device with a sensor attached to the umbilical cord clamp. The housing of the cord clamp includes an orifice to receive “a means for measuring physiological data” which is shown attached by a wire to a separate output device.

### SUMMARY

The existing designs do not provide real-time information immediately following the birth of a baby for the efficient monitoring and use by a medical team working to provide immediate, emergency care to a newborn. Embodiments of the present disclosure provide relevant information in the immediate area of the medical team without requiring ancillary components. Embodiments of the design may also be used in other emergency situations to provide real-time data directly to the medical team working on a patient.

The present disclosure provides biometric reading devices, systems and methods of use. The devices may be designed as stand-alone products or as a system of networked components. Devices read one or more physiological features of a user and provide physiological data based on the collected information.

Embodiments of a device may include a patch that can be attached to a patient with one or more biometric sensors and one or more display features. The biometric sensor may include one or more electrodes used to determine a heartrate through an analysis of electrical signals detected in the user's body. In some embodiments, the biometric sensor is a pulse oximeter. The biometric sensor may include one or more of electrodes, pulse oximeters, a glucose sensor, a respiratory sensor, a thermometer or another sensor device.

Embodiments of the device may include one or more display features including an LED light strip, a display screen operable to show alpha-numeric data waveforms and/or other information. Display features may be incorporated into a single display element and/or multiple display elements. In some embodiments, the device also includes an audible output device.

Embodiments of the device may include a computer module to control the operation of the device, analyze biometric data, store data and/or communicate data with another device. Some embodiments may include a user input to allow a user to start the device, control features of the device and/or stop operation of the device.

Some embodiments of the device may be part of a system that sends data to an external device. For example, embodiments of the device may transmit output signals to an external monitor and/or speaker for output. Some embodiments may send the data to a person's smart phone, tablet or other mobile device.

Embodiments of another device may include an umbilical cord biometric reading device. Embodiments of the umbilical cord device may include an umbilical cord strap that wraps around the infants umbilical cord, an output housing and a lead section that connects the strap to the housing. Sensor components may be located within the strap design such as pulse oximetry sensors, ECG electrode sensors and/or other sensors. The sensors may be connected to the housing through one or more wires within the lead section. Some embodiments of the housing include visual display features, such as colored lights, alpha-numeric outputs and/or waveform outputs. Within the housing, a computer module may be included some embodiments. The computer module may control the operation of the device, analyze biometric data, store data and/or communicate data with another device.

Embodiments of the system may include one or more embodiments of the biometric reading device and other external elements. Some embodiments of the system include a patch biometric reading device and an umbilical cord biometric reading device. The devices in some embodiments of the system operate to communicate in real time to share data. For example, the umbilical cord biometric reading device may transmit a heartrate and blood oxygenation reading to the patch device. In such embodiments, the patch device may take the data from the umbilical cord biometric reading device and analyze it with data obtained by the patch device to determine current biometric readings. In some embodiments, the patch may display the data on an integrated display or provide another output. Some embodiments may communicate with multiple external sensors and other devices to obtain additional data that may include redundant data to improve and/or confirm system accuracy in assessing biometric readings for a person.

In some embodiments, multiple external outputs and computer systems are integrated to output, review, assess and/or record the data from one or more biometric reading devices, such as a monitor, a smart phone, local or remote computer system and/or a server.

Some embodiments include a kit containing a biometric reading device and a wipe or other cleaning item to prepare a person's skin for application of the biometric reading device. The kit is sealed within a sanitary package prior to use in some embodiments.

Some embodiments of the method include placing a biometric sensor device on a newborn at the time of birth, sensing electrical signals produced by newborn, determining a heartrate of the newborn based on the sensed electrical signals, displaying an indication of the heartrate on the device. The display may provide a numerical indication, a wave form indication and/or a color coded indication. In some embodiments, the display is replaced with an audible output. A medical professional may use the heartrate indication as a factor in determining appropriate medical care for the newborn.

In some embodiments, the method may include utilizing a pulse oximetry sensor to determine blood oxygenation and displaying an indication of the blood oxygenation data. In addition, the pulse oximetry sensor in such an embodiment may be used to provide a secondary heartrate reading which may be used to evaluate an accurate heartrate of the baby.

Embodiments of the method may also include receiving additional data from other sensors placed on the newborn. The additional data may be used in evaluating the biometrics in some embodiments. In some embodiments, the additional data may be output on the display for the medical professional to see.

The data may be transmitted to another device for further analysis, storage and/or output in some embodiments. For example, the heartrate and oxygenation data may be transmitted to a nearby computer for evaluation.

### A BRIEF DESCRIPTION OF THE DRAWINGS

Embodiments will now be described, by way of example only, with references to the accompanying drawings in which:

FIG. 1 is an exploded view of an embodiment of a biometric sensing patch;

FIG. 2 is an exploded view of another embodiment of a biometric sensing patch;

FIG. 3 depicts an embodiment of a biometric sensing system;

FIG. 4 depicts another embodiment of a biometric sensing system;

FIG. 5 is a block diagram of another embodiment of a biometric sensing patch;

FIG. 6 depicts another embodiment of a biometric sensing system placed on a newborn;

FIG. 7 depicts an embodiment of a biometric sensing device;

FIG. 8 depicts another embodiment of a biometric sensing patch; and

FIG. 9 is a perspective view of an embodiment of a kit including a biometric sensing patch.

### DETAILED DESCRIPTION

While this invention may be embodied in many different forms, there will herein be described in detail preferred embodiments of the invention with the understanding that the present disclosure is to be considered as an exemplification of the principles of the invention and is not intended to limit the broad aspects of the invention to the embodiments illustrated. It will be understood that the invention may be embodied in other specific forms without departing from the spirit or central characteristics thereof. The present embodiments, therefore, are to be considered in all respects as illustrative and not restrictive, and the invention is not to be limited to the details given herein.

The devices, systems and methods described herein may typically be used by a medical professional on a patient in a medical setting. For purposes of discussion, the user herein will often be referred to as a medical professional and/or patient as applicable; however, other users may also utilize the devices, systems and methods described herein. In addition, users may utilize the devices, systems and methods described herein in non-medical settings without deviating from the scope of the disclosure.

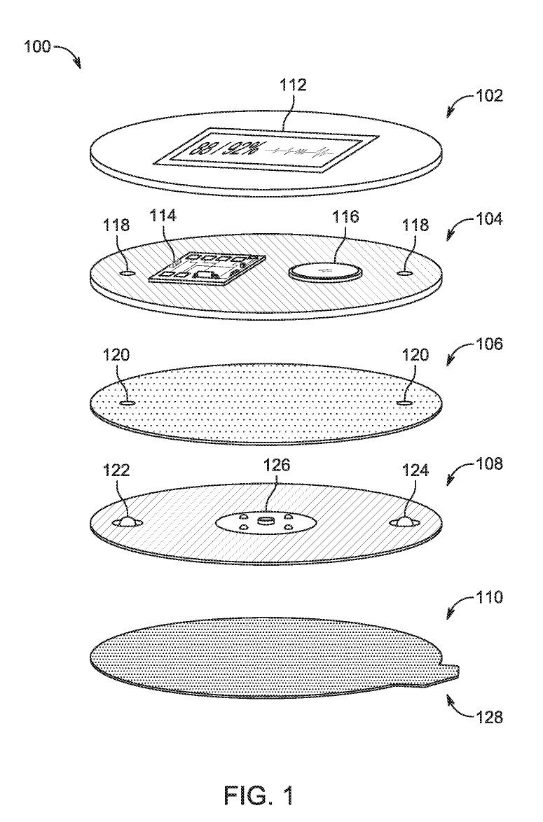

FIG. 1 shows an embodiment of a biometric sensing patch 100 in an exploded view. In the embodiment shown, the patch 100 includes five layers including a display layer 102, a substrate layer 104, an insulation layer 106, a sensor layer 108 and a liner layer 110. Some embodiments of the biometric sensing patch 100 may include fewer or more layers. For example, the insulating layer 106 may be combined with the substrate layer 104 and/or the sensor layer 108 instead of being a separate layer. For another example, the substrate layer 104 and the display layer 102 may be combined in some embodiments. For yet another example, a top liner layer may be applied over the display layer 102.

The layers of the patch 100 are constructed of flexible materials to allow the patch 100 to flex when applied to a patient and form to the patient. The flexibility of the patch 100 also allows the patch 100 to stay attached and operational while the patient is moving. Accordingly, one skilled in the art in view of this application will recognize that the display layer 102, the substrate layer 104, the insulation layer 106 and the sensor layer 108 combined will have the flexibility to fit intended patients and allow natural range of motion for the patient at the intended location for placement. For example, when the patch 100 is intended for use on a newborn's chest, the flexibility of the patch 100 will allow for a range of newborn body types and the natural movement of a newborn's chest. For another example, when the patch 100 is intended to be used on an adult female's chest, the flexibility of the patch 100 will allow for a range of women's body types and the natural movement of a woman's chest.

The layers of the patch 100 may be constructed from a variety of materials including foams, woven fabrics, rubbers, plastics, silicones and/or other materials. As discussed further herein most of the layers within the patch 100 will be constructed, at least in part, with non-conductive materials.

The upper layers of the patch 100 including the display layer 102, the substrate layer 104, the insulation layer 106 and the sensor layer 108 are combined to form the operational portion of the patch 100 while the liner layer 110 is designed to be removed prior to placement on a patient exposing the sensor layer 108 for contact with the patient's skin. The upper layers 102, 104, 106 and 108 of the patch 100 are attached and have a connection that allows the patch 100 to be fluid impermeable or at least fluid resistant. Alternative embodiments may include internal components that are safe to operate in a fluid environment obviating the need for the entire patch 100 to be fluid resistant. In some embodiments, all four of the display layer 102, the substrate layer 104, the insulation layer 106 and the sensor layer 108 may be connected. In some embodiments, the display layer 102 and the sensor layer 108 may be connected to each other with the substrate layer 104 and the insulation layer 106 fitted within the display layer 102 and the sensor layer 108. The layers may be attached using mechanical techniques (e.g. press seals, heat seals, etc.), adhesives and/or other connections.

In addition, the patch 100 may include one or more gel layers applied to all or portions of a display layer 102, a substrate layer 104, an insulation layer 106, a sensor layer 108 and a liner layer 110. Some of the gel layers may include fluid repelling features and operate in conjunction with the connections to make the patch 100 fluid resistant. Some of the gels may be conductive to improve the electronic connections. For example, a conductive gel may be applied to the bottom of the sensor layer 108 to improve the sensor connections with the patient's skin.

The display layer 102 is the top outer layer of the patch 100 and includes a display 112 in this embodiment. The display 112 is depicted in this embodiment with an alpha-numeric output and a waveform output. The display 112 may be any type of visual output element, including but not limited to a digital display, an LED array, an analog display feature and/or any other display element or combination of elements. For example, the display 112 may be an array of LEDs that provide specific color outputs to indicate different information. In some embodiments, the display 112 is designed from flexible material that will flex with the patch 100. In some embodiments, the display 112 may not have the same flexibility characteristics as the remainder of the display layer 102.

In some embodiments, alternative designs of the display 112 may be used. For example, the display 112 may extend to the edges of the display layer 102. For another example, the display 112 may include a secondary display element that extends from the frame of the illustrated digital display to the edge of the display layer 102. In such embodiments, the digital display portion may show alpha-numeric and/or waveform outputs while the secondary display element shows colors indicative of medical guidelines associated with the biometric data identified by the device.

The substrate layer 104 includes a computer module 114 and a power source, shown as battery 116. The computer module 114 includes elements of a computer including but not limited to a processor, memory and input/output elements. In some embodiments, the computer module 114 may include communication elements to receive and/or transmit signals with other external devices.

Passages 118 are also shown in the substrate layer 104 to illustrate optional passages for wiring or other elements to connect elements in the sensor layer 108 to the substrate layer 104 elements including the computer module 114 and battery 116. While passages 118 are included for illustrative purposes, an assembled patch 100 would include wiring or other connections needed to operably connect the operational elements in to one another. One skilled in the art would further recognize in light of the application that the size of passages 118 may vary depending on the connections used. For example, the passages 118 may seal against wires passing through the passages 118. In addition, while two passages 118 are shown, there may be fewer or more passages in some embodiments of the patch 100.

The computer module 114 controls the operation of the patch 100 through receiving data from biometric sensor components, such as those located in the sensor layer, evaluating the received data to determine biometric information and providing the applicable output to the display 112 for communication to observer of the patch 100. The actual output may be based upon standardized requirements stored in the memory of the computer module 114. For example, the computer module 114 may direct the display 112 to provide specific color outputs correlated to the calculated heartrate of the patient, such as a red output for a heartrate under 60 beats per minute, a yellow output for a heartrate from 60 to 100 beats per minute and a green output for a heartrate of 100 beats per minute or higher.

In some embodiments, the substrate layer 104 is a flexible backing material and the computer module 114 is a flexible circuit board attached to the backing material. The battery 116 may also be comprised of a semi-flexible material and attached to the backing material of the substrate layer 104. In some embodiments, the computer module 114 is integrated into the material of the substrate layer 104 or placed on the backing material as a thin film.

In some embodiments, the battery 116 is designed to have a power capacity correlated to the intended use of the patch 100. For example, when the patch 100 is a disposable device intended to stay on the patient for 24 hours, the capacity of the battery 116 may allow for the patch 100 to be operable for 24 hours. In some embodiments, the capacity may be designed to include additional capacity to allow operation for a specific amount of overuse. This additional capacity may also operate as a cushion to limit the likelihood of an underpowered device.

The insulation layer 106 includes passages 120 that correspond to the passages 118 in the substrate layer 104. The passages 120 also illustrate optional passages for wiring or other elements to connect elements in the sensor layer 108 to elements in the substrate layer 104. While passages 120 are included for illustrative purposes, an assembled patch 100 would include wiring or other connections needed to operably connect the operational elements in to one another. One skilled in the art would further recognize in light of the application that the size of passages 120 may vary depending on the connections used. For example, the passages 120 may seal against wires passing through the passages 120. In addition, while two passages 120 are shown, there may be fewer or more passages in some embodiments of the patch 100.

The insulation layer 106 provides insulation between the substrate layer 104 and the sensor layer 108. In some embodiments, the insulation layer 106 limits the likelihood of interference between the elements of the substrate layer 104 and the sensing components located in the sensor layer 108. For example, the insulation layer 106 prevents electrodes 122 and 124 from sensing electrical signals within the computer module 114. In addition, the insulation layer 106 may prevent ancillary light from interfering with a light sensor within pulse oximeter 126 in some embodiments.

In some embodiments, the insulation layer 106 is integrated into one or both of the substrate layer 104 and the sensor layer 108. For example, the backing material of the substrate layer 104 may also comprise an insulating material. For another example, the sensor elements in sensor layer 108 may be embedded in the bottom of an insulation material. In some embodiments, the sensor elements may not require additional insulation from other components, and the insulation layer 106 may not be included.

In this embodiment, the sensor layer 108 includes electrodes 122 and 124 and a pulse oximeter 126. During operation, the electrodes 122 and 124 are used to detect electrical signals within the patient's body that indicate heart activity. The computer module 114 receives readings from the electrodes 122 and 124 and analyzes the readings to determine a patient's heartrate.

In this embodiment, the pulse oximeter 126 is shown as a reflective pulse oximeter with LEDs and an optical sensor to detect reflected light emitted from the LEDs. The detected light data is received by the computer module 114 and analyzed to determine oxygenation and/or a heartrate. In some embodiments, the pulse oximeter 126 may be another type of pulse oximeter.

In some embodiments, the sensor layer 108 may include a different array of sensors. Some embodiments may only include one or more electrodes 122, 124 to detect heart activity. Other embodiments may include the electrodes 122, 124, the pulse oximeter 126, a thermometer, a glucose sensor, a respiratory sensor and/or other sensors.

The liner layer 110 covers the bottom of the sensor layer 108 prior to use of the patch 100. In this embodiment, the liner layer 110 includes a tab 128 to provide place to grip when removing the liner layer 110 from the remainder of patch 100. The liner layer 110 may help seal a gel against the bottom of the sensor layer 108 and prevent the gel from drying out prior to use.

When the patch 100 is used, the medical professional will grab tab 128 and remove the liner layer 110 from the bottom of the patch 100. In some embodiments, removing the liner layer 110 may also turn on the patch 100. Once the liner layer 110 is removed, the medical professional will place the bottom of the patch 100 (bottom of sensor layer 108) against the patient's skin. For example, the patch 100 may be placed on the chest of a newborn immediately after birth. Once the patch 100 is placed on the patient, the computer module 114 will begin receiving data from the electrodes 122, 124 and the pulse oximeter 126. The computer module 114 will analyze the data received and determine a heartrate. Once the heartrate is determined, the computer module 114 will output the result to the display 112. The computer module 114 may also output the heartrate waveform to the display 112. In some embodiments, the computer module 114 will also determine an oxygenation level and output the oxygenation percentage result to display 112.

During operation of the patch 100 on a newborn, medical professionals can review the relevant data on the display 112 which is located on the chest of the newborn on which the professional is attending. Providing the display 112 on the chest of the patient allows the medical professional to remain focused on the newborn while also monitoring the biometric data relevant to potential emergency situations. Accordingly, the patch 100 may allow the medical professionals to act faster in an emergency situation wherein every second can impact the life of the child because the medical professionals are not required to look away from the patient to receive the biometric data. In addition, the medical professionals would not have to include additional staff separately monitoring the biometric data and repetitively stating the relevant biometric data which may interfere with other emergency communications occurring at the same time.

Embodiments of the patch 100 may be placed at a variety of locations against the patient's skin, including the chest, forehead and other locations, that allow for the patch to operate as described herein. As discussed herein, the patch 100 may be placed at a location that allows the medical professional to remain focused on the patient while also monitoring the biometric data relevant to potential emergency situations. For example, the patch 100 may be placed on a patient's forehead and would be readily observable.

Additional data may also be evaluated and displayed on the display 112 in some embodiments. For illustrative purposes, the display 112 shows a heartrate of 88, an oxygenation of 92% and an electrical activity waveform. Alternative embodiments of the patch 100 may provide more or less information. In some embodiments, the computer module 114 may allow user control to select the preferred output options.

In some embodiments, the computer module 114 directs the display 112 to provide a color output indicative of the heartrate relative to medical guidelines stored in the memory of the computer module 114. For example, the medical guidelines for neonatal resuscitation immediately following birth may be stored in the memory and an indicative color is assigned for each heartrate range provided in the guidelines. When the computer module 114 receives data from the electrodes 122, 124 and/or the pulse oximeter 126, the computer module 114 determines the heartrate and then compares the heartrate to each of the heartrate ranges defined by the stored medical guidelines. Once the applicable range is determined by the computer module 114, the computer module 114 causes the display 112 to output the indicative color associated with the applicable heartrate range. For example, the display 112 may be red for a heartrate under 60 beats per minute, yellow for a heartrate from 60 to 100 beats per minute and green for a heartrate of 100 beats per minute or higher. In some embodiments using a color coded output, the computer module 114 will cause the display 112 to blink at the actual heartrate or otherwise indicate the actual heartrate within the given range.

In some embodiments, the patch 100 may also incorporate failsafe mechanisms. For example, the patch 100 may utilize redundant sensors to increase the likelihood of successfully obtaining a biometric readings and calculating the heartrate based upon each of the successfully obtained readings to provide increased accuracy in determining biometric characteristics of the patient.

In addition, some embodiments will provide error evaluation and notification features. For example, the patch 100 may include redundant and/or alternative sensors for obtaining readings associated with a characteristic of the patient. During an evaluation of the sensor readings, the computer module 114 may determine that one or more of the readings may be in error. If the correct result cannot be determined due to the error, the computer module 114 may output an error message and/or indication to ensure the medical professional can take appropriate steps in view of the error.

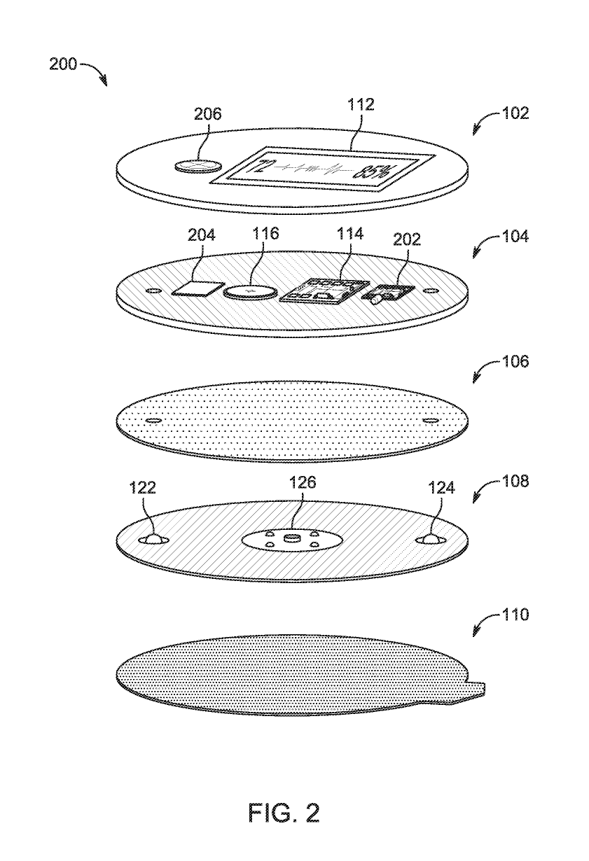

FIG. 2 shows another embodiment of a biometric sensing patch 200 in an exploded view. Similar to FIG. 1, the patch 200 includes five layers including the display layer 102, the substrate layer 104, the insulation layer 106, the sensor layer 108 and the liner layer 110. Some embodiments of the biometric sensing patch 200 may include fewer or more layers. As discussed above, the layers of the patch 200 are constructed of flexible materials to allow the patch 200 to flex when applied to a patient and form to the patient. The layers of the patch 200 may be constructed from a variety of materials including foams, woven fabrics, rubbers, plastics, silicones and/or other materials. As discussed further herein most of the layers within the patch 200 will be constructed, at least in part, with non-conductive materials.

While aspects of the layers of patch 200 are described in further detail with regard to FIG. 1, the embodiment shown in the patch 200 illustrates additional and/or alternative aspects of a biometric device. In addition to elements discussed elsewhere herein, the patch 200 includes a communication module 202, a switch 204 and a button 206. The patch 200 also illustrates another arrangement of the elements of in a biometric sensing device.

The display layer 102 of the patch 200 includes display 112 and a button 206 in this embodiment. The display 112 is depicted in this embodiment with an alpha-numeric output and a waveform output. This display 112 shows the output of data in an alternative arrangement with the “72” indication on one side of a waveform and an 85% reading on the opposite side.

The substrate layer 104 includes the computer module 114 the power source (shown as battery 116), communication module 202 and switch 204. The button 206 in conjunction with switch 204 operates as a control of the patch 200. In some embodiments of the patch 200, the button 206 comprises a flexible material that may be compressed actuating the switch 204. The flexible material of button 206 may be formed from the same piece of material as the display layer 102.

In some embodiments, the button 206 and the switch 204 operate solely as an on/off control. In other embodiments, the button 206 and the switch 204 may also facilitate control of one or more features of the patch 200. For example, pressing the button 206 may allow the user to select a preferred display option, such as only showing the heartrate and oxygenation level without any waveform.

As discussed above, the computer module 114 controls the operation of the patch 200 through receiving data from biometric sensor components, such as those located in the sensor layer 108, evaluating the received data to determine biometric information and providing the applicable output to the display 112 for communication to observer of the patch 200. The actual output may be based upon standardized requirements stored in the memory of the computer module. For example, the computer module 114 may direct the display 112 to provide specific color outputs correlated to the calculated heartrate of the patient, such as a red output for a heartrate under 60 beats per minute, a yellow output for a heartrate from 60 to 100 beats per minute and a green output for a heartrate of 100 beats per minute or higher.

In some embodiments, the computer module 114 may be off until the button 206 is pressed actuating the switch 204 and turning the computer module 114 on. When the computer module 114 is turned on, the computer module 114 will begin operation of the additional patch elements. In some embodiments, the computer module 114 will initiate an internal clock feature when the device is turned on. Some embodiments may include an option to display the clock data on display 112.

The communication module 202 operates to transmit and/or receive (collectively “communicate”) data wirelessly with external devices—such as a monitor, a smartphone, a tablet, another sensor, etc. Some embodiments of the communication module 202 are near field communication modules allowing communications with devices within a short range of the patch 200. Near field communication modules may operate on the BLUETOOTH communication standard, the ZIGBEE communication standard, and/or any other near field communication standard.

Some embodiments of the communication module 202 may provide for communication beyond the range of a near field communication module. For example, the communication module 202 may be a WIFI communication module, a cellular communication module or another wide area communication module.

In some embodiments, the substrate layer 104 is a flexible backing material and the computer module 114, switch 204 and the communication module 202 are flexible circuit boards attached to the backing material. The battery 116 may also be comprised of a semi-flexible material and attached to the backing material of the substrate layer 104. In some embodiments, the computer module 114, switch 204 and the communication module 202 are integrated into the material of the substrate layer 104 or placed on the backing material as a thin film.

In some embodiments, the communication module 202 communicates with other devices in real-time. Some embodiments of the communication module 202 may operate on a periodic basis. The operation of the communication module 202 may be managed or modified from a default setting by computer module 114. For example, the computer module 114 may cause the communication module 202 to change from real-time communication to a periodic communication pattern to lower power consumption and thereby extend the life of battery 116. For another example, the computer module 114 may cause the communication module 202 to transmit pairing signals periodically to identify whether another device is ready to pair with the patch 200 and will initiate real-time communication only upon receiving the corresponding pair signal from an external device. One having ordinary skill in the art will understand that a pairing process may include one or more security measures, such as authorization checks and/or encryptions, to ensure the data communicated is to and from an approved external device.

When the patch 200 is used, the medical professional may actuate the button 206 and remove the liner layer 110 from the bottom of the patch 200. The patch 200 may begin operations, such as starting the display 112, initiating communications through communication module 202 and/or other operations, as soon as the button 206 is pressed. Once the liner layer 110 is removed, the medical professional will place the bottom of the patch 200 (bottom of sensor layer 108) against the patient's skin. Once the patch 200 is placed on the patient, the computer module 114 will begin receiving data from the electrodes 122, 124 and the pulse oximeter 126. In some embodiments, the computer module 114 may also receive additional data through the communication module 202. The computer module 114 will analyze the data received and may determine a heartrate, oxygenation percentage and/or additional biometric data. Once the biometric data is determined, the computer module 114 will output the result to the display 112. The biometric data may be displayed in an alphanumeric format, a waveform format and/or another format—e.g. a color coded format, a medical reference code, an estimated APGAR reference, etc.

In some embodiments, the patch 200 is designed for use on a newborn to evaluate the health characteristics of the child in the moments following birth. The device may be designed for operation beginning at the time of birth and for a period immediately following the birth. In the delivery room, a medical professional may press the button 206 at the time of birth to turn on the patch 200. In addition, the button 206 may begin a clock feature within the computer module 114 to provide the time from birth. Some medical guidelines and procedures are based in part upon the time from birth. For example, the target oxygenation percentage is correlated to the time from birth within some medical guidelines.

In addition, a medical professional may observe the APGAR characteristics at specific times after the birth of a child. For example, the medical professional may evaluate the infant's APGAR score at the one minute, five minute and ten minute marks. In some embodiments, the computer module 114 may provide timing outputs to the display 112, such as a warning light, a flashing time indicator, etc., to correspond with the selected times for evaluating the APGAR score or other trigger times.

As soon as practical following the birth of the child, a medical professional will remove the liner layer 110 from the bottom of the patch 200 and apply the patch to the child's chest. In some embodiments, a gel or other substance may be exposed on the bottom of the patch 200 that facilitates a connection to the child's skin. The gel or other substance located at the electrodes 122 and 124 may have conductive properties to allow the electrodes 122 and 124 to read electrical signals produced by the heart. In some embodiments, the gel or substance is designed to facilitate a connection prior to removal of a vernix coating or other biological material on the child. In some embodiments, a medical professional will first wipe vernix and/or other biological material from the location where the patch 200 will be placed.

Once the patch 200 is placed on the newborn, the patch 200 will gather data from the electrodes 122 and 124, the pulse oximeter 126, any additional sensors and/or any external data sources in communication with the patch 200. The computer module 114 will evaluate relevant data to determine biometric information about the baby, such as a heartrate and an oxygenation percentage. The computer module 114 will output relevant results, or indications thereof, on the display 112. In addition, the computer module 114 may transmit data and/or evaluation results to an external device via the communication module 202.

During operation of the patch 200 on a newborn, medical professionals can review the relevant data on the display 112 which is located on the chest of the newborn on which the professional is attending. Providing the display 112 on the chest of the patient allows the medical professional to remain focused on the newborn while also monitoring the biometric data relevant to potential emergency situations.

In some embodiments, the display 112 may provide a color output indicative of the heartrate relative to medical guidelines stored in the memory of the computer module 114. Providing the indicative color may allow the medical professional to recognize relevant information without modifying their focus from other medical activities for the newborn. For example, a medical professional may notice the display 112 change from yellow to green indicating that the newborns heartrate improved to a safer level.

In some embodiments, the display 112 or an external display connected wirelessly to the patch 200 via the communication module 202 may also show a number or indication correlated to one or more aspects of an APGAR score. One skilled in the art will recognize that APGAR scoring is a simple assessment to quickly gauge the status of a newborn and the newborn's response to resuscitation or other procedures. The APGAR score is based upon a medical professional's review of five characteristics of the newborn, including the color, heartrate, reflexology, muscle tone and respiration. In some embodiments, the patch 200 may determine a preliminary result correlated to one or more characteristics of the APGAR score. For example, the patch 200 may provide a preliminary score for the heartrate, respiration and color based upon sensor analysis tied to the heartrate sensors, oxygenation sensors, respiration sensors and/or other sensors. The medical professional may use the preliminary score from the patch 200 along with observed scores for other characteristics to determine an APGAR score. The accuracy and completeness of an APGAR or other status indicator may be improved based upon scientific studies of data collected with one or more patches and correlated to various health outcomes.

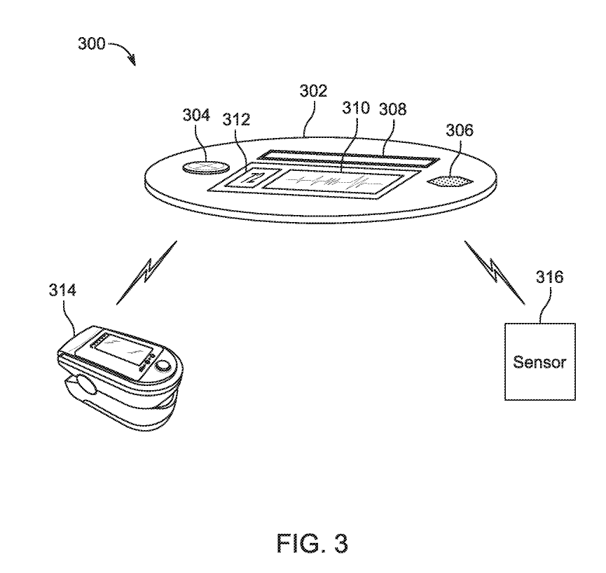

FIG. 3 depicts an embodiment of a biometric sensing system 300 including a patch 302, a pulse oximeter 314 and another sensor 316. The patch 302 is similar to patches 100 and 200 discussed previously and includes similar internal features, such as electrode sensors, a computer module, a power source and/or other elements. The patch 302 may also include additional elements that are not shown in this view, such as alternative sensors.

The display surface of patch 302 in this embodiment includes a button 304, an audible output 306, a bar display 308, a waveform display 310 and an alphanumeric display 312. The button 304 may operate as an on/off control and/or a feature control. Embodiments of the button 304 may be similar to the button 206 described with respect to FIG. 2.

The audible output 306 may be a speaker or any other audible output device. During operation the audible output 306 may be used to provide information to the user of the patch 302. For example, the audible output 306 may provide an audible noise correlated to each heartbeat detected by the patch 302. For another example, the audible output 306 may provide voice outputs stating relevant data. One having ordinary skill in the art will recognize that the audible output 306 may be used to provide information regarding the data obtained or received by the patch 302, results of an analysis by the patch 302, status information regarding the patch 302 or connected devices and/or other information.

In addition to the audible output 306, this embodiment of the patch 302 includes multiple display features—a bar display 308, a waveform display 310 and an alphanumeric display 312. While this embodiment includes multiple separate display features, other embodiments, such as those described elsewhere herein, may only include one display feature. In addition, certain display features may be operable to provide multiple display types in separate sections of a single display.

The bar display 308 may provide an indication for biometric readings. In some embodiments, the bar display 308 may provide a color indication correlated to a medical guideline. For example, the bar display 308 may be red for a heartrate under 60 beats per minute, yellow for a heartrate from 60 to 100 beats per minute and green for a heartrate of 100 beats per minute or higher. In some embodiments using a color coded output, the bar display 308 will blink at the actual heartrate or otherwise indicate the actual heartrate within the given range. In some embodiments, the bar display 308 may light up from one end to the other to illustrate a gauge. For example, the lit portion of the bar display 308 may begin on the left and extend to a point in the bar display 308 representative of the current heartrate of the patient. The lit portion of the bar display 308 grows as the patient's heartrate increases and shrinks as the patient's heartrate decreases.

The waveform display 310 shows a heartrate waveform in this embodiment. In some embodiments, the waveform display 310 may provide alternative information. For example, the waveform display 310 may show pulse readings obtained by the pulse oximeter 314. For another example, the waveform display 310 may show respiration patterns based upon data received from the sensor 316.

The alphanumeric display 312 may show any alphanumeric output in this embodiment. For example, the alphanumeric display 312 may show the calculated heartrate of the patient. For another example, the alphanumeric display 312 may show an oxygenation percentage, a temperature and/or other information obtained by the patch 302, the pulse oximeter 314, the sensor 316 and/or another source of information.

The outputs including displays 308, 310 and 312 and audible output 306 may provide information regarding the data obtained or received by the patch 302, results of an analysis by the patch 302, status information regarding the patch 302 or connected devices and/or other information. The outputs may be coordinated to provide the same or different information depending on settings of the patch 302.

In some embodiments, the button 304 may be operable to facilitate the selection of settings for the patch 302. In some embodiments, one or more of the bar display 308, the waveform display 310 and the alphanumeric display 312 may be an input device, such as a touch screen control, that may be used to control one or more features of the patch 302. In some embodiments, the audible output 306 may include a microphone to record sounds and/or facilitate voice activated controls for the patch 302.

In this embodiment, the patch 302 is wirelessly connected to pulse oximeter 314 through wireless modules in patch 302 and pulse oximeter 314. The pulse oximeter 314 is depicted as a fingertip or clip pulse oximeter in this embodiment. One having ordinary skill in the art will recognize that other types of pulse oximeters may be used in place of the fingertip design. During operation, the pulse oximeter 314 obtains data regarding an oxygenation level of the user and may also obtain data regarding the patient's heartrate. In some embodiments, the pulse oximeter 314 communicates the obtained data directly to the patch 302 in real-time or near real-time. In some embodiments, the pulse oximeter 314 may include a processor or other computer module to determine an oxygenation percentage and/or a heartrate and sends calculated information to the patch 302.

In addition, the patch 302 is also wirelessly connected to another sensor 316 through wireless modules in patch 302 and the sensor 316. The sensor 316 may be any type of sensor that may provide relevant data to the patch 302, including one or more of electrode sensors, pulse oximeters, a glucose sensor, a respiratory sensor, a thermometer or another sensor device. The sensor 316 may be applied to the patient to obtain additional data that is distinct from the data obtained by the patch 302. For example, the sensor 316 may be a temporal thermometer attached to the patient's head. For another example, the sensor 316 may be a glucose sensor attached to an infant's umbilical cord. In addition, the sensor 316 may obtain data redundant to the data obtained by the patch 302. For example, the sensor 316 may be another electrode sensor designed to obtain a heartrate whereby the patch 302 may compare data to ensure the results are properly correlated. If the redundant results are not properly correlated, the patch 302 may indicate a warning to let the medical professional know a potential error may have occurred.

During operation of the system 300, one or more medical professionals may work together to properly apply the patch 302, the pulse oximeter 314 and the sensor 316 to a patient. The medical professional(s) would ensure the devices are each turned on (e.g. button 304 is pressed to turn on the patch 302) and may wait for an indication that the devices have all properly paired together. Once the devices are communicating and properly attached, data is received by the patch 302 and processed to determine and output information to the medical professional(s) via at least one of the outputs.

In some embodiments, the system 300 may be used to conduct a critical congenital heart disease screening using two of the pulse oximeters 314. The first pulse oximeter 314 may be placed on the infant's right hand to acquire a preductal reading and the second pulse oximeter 314 may be placed on either one of the infant's feet to acquire a post-ductal reading. During operation for a critical congenital heart disease screening, the system 300 will acquire preductal and post-ductal heartrate and oxygen saturation data from the two pulse oximeters 314. The data may be sent to the patch 302. In some embodiments, the patch 302 will show the data directly on one or more of displays 308, 310 and 312. In other embodiments, the computer module within patch 302 will analyze the data and provide relevant results (such as the preductal oxygenation percentage, the post-ductal oxygenation percentage, the oxygenation percentage difference between the preductal and post-ductal readings, a pass/fail indication based upon screening guidelines and/or another relevant result) the on one or more of displays 308, 310 and 312. For example, the display 308 may light up with a color to indicate the results of the screening, such as green to indicate the preductal and post-ductal oxygenation levels are each at or above 95% and within 3% of each other, yellow to indicate the results are within a set deviation for a passing result (e.g. the preductal and/or post-ductal oxygenation levels are below 95% and at or above 90% and/or the preductal and/or post-ductal oxygenation levels are not within 3% of each other) and red to indicate the preductal and/or post-ductal oxygenation levels are below 90%.

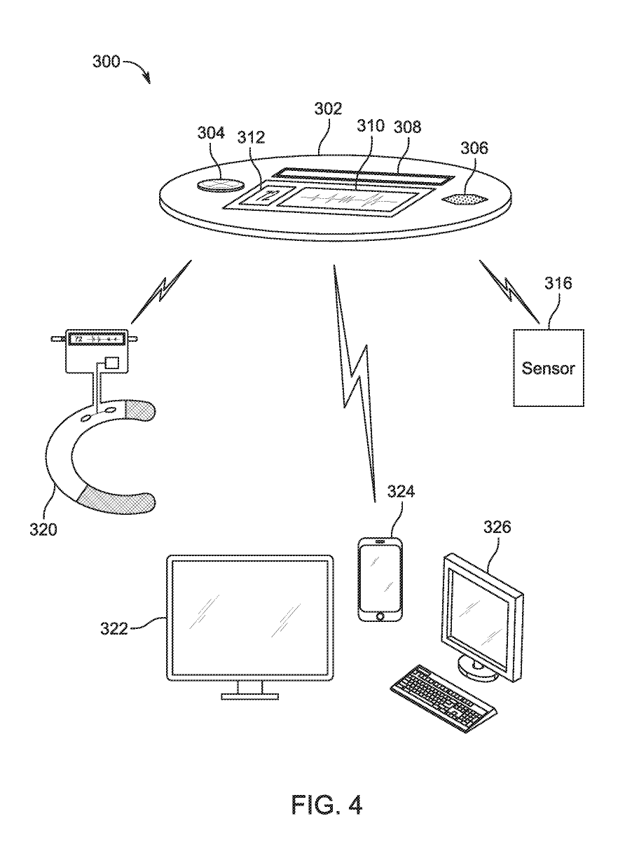

FIG. 4 depicts another embodiment of the biometric sensing system 300. In this embodiment, the biometric sensing system 300 includes the patch 302 and sensor 316 discussed above. In addition to these components, the biometric sensing system 300 includes a pulse oximeter 320 (illustrated as a different type of pulse oximeter than pulse oximeter 314), a display monitor 322, a smartphone 324 and a computer 326. In the biometric sensing system 300, each of the devices is wirelessly connected to the patch 302 and/or each other. In some embodiments, the system 300 may include a hub component to facilitate communications between devices. In such an embodiment, the hub may also facilitate relaying communications to other devices and/or systems that may be remote from one or more of the devices shown and beyond the range of their communication capabilities.

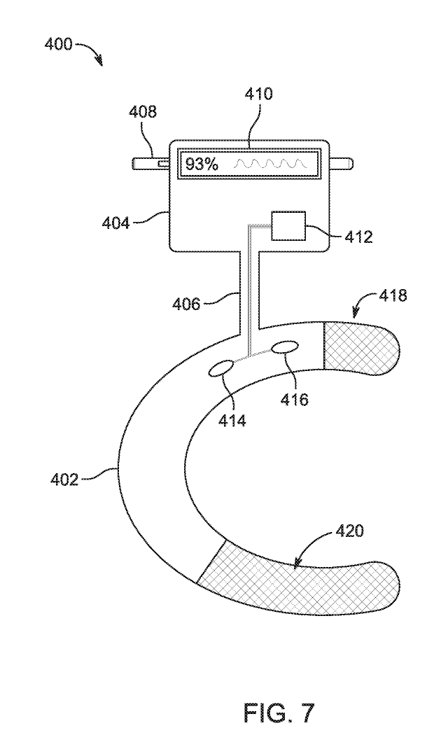

The pulse oximeter 320 is an umbilical cord pulse oximeter that includes its own output features. During operation, the pulse oximeter 320 obtains data regarding an oxygenation level of the user and may also obtain data regarding the patient's heartrate. In some embodiments, the pulse oximeter 320 communicates the obtained data directly to the patch 302 in real-time or near real-time. In some embodiments, the pulse oximeter 320 sends calculated information regarding the oxygenation percentage and/or the heartrate to the patch 302. Additional elements of the pulse oximeter 320 are discussed further in the context of FIG. 7.

The monitor 322 in this embodiment may operate as an additional and/or a redundant display for the patch 302. The larger monitor 322 may be capable of providing more information and detail regarding the patient data and other potentially relevant data. For example, the monitor 322 may include a running clock to show the time from birth, the calculated heartrate and electrical waveform, the calculated oxygenation percentage and pulse waveform, the patient's temperature and other information. While the monitor 322 may be available for reference, the medical professional does not have to use the monitor 322 during emergency situations because the pertinent information is provided on the display outputs on the patch 302.

This embodiment of the system 300 shows smartphone 324 as another connected element within the network of devices. One having ordinary skill in the art will recognize that alternative devices may be included in the network of devices of system 300 in addition to or instead of those shown in FIG. 4, including tablet devices, laptop devices and/or any other mobile computing device. In some embodiments, analog devices, such as phones and pagers, may be able to operate within the system 300 through texting protocols. These options allow for the system 300 to be utilized in a wide variety of environments including those away from hospitals and other medical facilities. For example, a person working to help others in a remote village may be able to use the patch 302 with a smartphone 324 with limited additional supplies and/or equipment.

The smartphone 324 in this embodiment may also operate as an additional and/or a redundant display for the patch 302. In addition, the smartphone 324 may provide additional medical information, such as applicable medical guideline information, that may be available as a reference for the medical professional. In some embodiments, the smartphone 324 may include application software that allows the app to identify relevant medical information and correlate the data to the medical information. The application may also allow a user to select relevant information to find out additional information supporting a diagnosis, a guideline or procedure.

The smartphone 324 may also facilitate additional outputs for data, warnings and/or other information. For example, the smartphone 324 may provide audible and/or mechanical outputs (e.g. vibration) to indicate a heartrate or other information. In addition, the smartphone 324 may use alternative communication means (e.g. cellular, WIFI, Internet connections, etc.) to communicate information to others in areas remote from the patient and patch 302. For example, the smartphone 324 may be designed to communicate a WIFI signal to a medical professional in another area of a medical facility to call for assistance and/or to provide updates. The operation of the smartphone 324 may depend on data thresholds. For example, the smartphone 324 may message a cardiologist in the event the heartrate is below a certain threshold.

This embodiment also includes the computer 326. The computer 326 may be a desktop or laptop computer, a server or any other type of computing device sufficient to facilitate a selected operation. For example, a desktop or laptop computer may facilitate many of the display and information features discussed in connection with the smartphone 324. For another example, a server may facilitate the aggregation, storage and/or analysis of data from multiple patients. In some embodiments, the server may be used to collect de-identified information from multiple patients for research and educational purposes.

In some embodiments, the computer 326 may receive and record data while the patch 302 is used. After the medical process is completed, the computer 326 may be used to conduct a review and analysis of the patient and the medical process. In addition, the computer 326 may be used for updating and completing a patient's medical records.

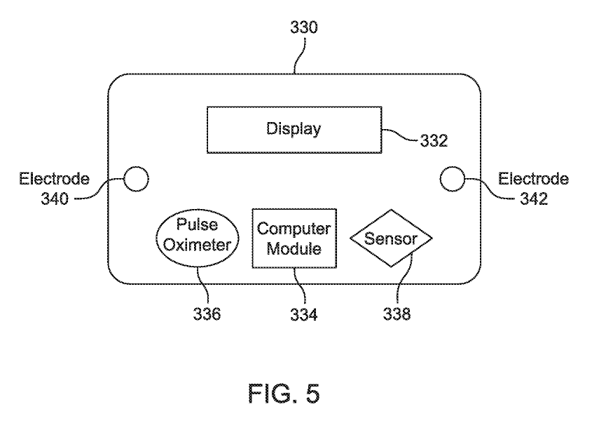

FIG. 5 shows a block diagram of an embodiment of a biometric sensing device 330. The biometric sensing device 330 includes a display 332, a computer module 334, a pulse oximeter 336, a sensor 338, a first electrode 340 and a second electrode 342. The biometric sensing device 330 may be a patch similar to those described elsewhere herein. In some embodiments, the biometric sensing device 330 may be part of a band or wrap. The biometric sensing device 330 may be embodied in other structural designs as well that may be tailored for a specific type of use.

The display 332 may operate in a similar manner to the other display elements described herein. The display 332 may be used to show data, calculated results and/or other information for the user of the biometric sensing device. In some embodiments, the display 332 may also operate as a user input element via touch screen operations or other control designs.

The computer module 334 includes elements of a computer including but not limited to a processor, memory and input/output elements. A power source may be included in the computer module 334 or separately within the biometric sensing device 330. In some embodiments, the computer module 334 may include communication elements to receive and/or transmit signals with other external devices. The computer module 334 controls the operation of the biometric sensing device 330 through receiving data from biometric sensor components, evaluating the received data to determine biometric information and providing the applicable output to the display 332 for communication to observer of the biometric sensing device 330. The actual output may be based upon standardized requirements stored in the memory of the computer module 334. For example, the computer module 334 may direct the display 332 to provide specific color outputs correlated to the calculated heartrate of the patient, such as a red output for a heartrate under 60 beats per minute, a yellow output for a heartrate from 60 to 100 beats per minute and a green output for a heartrate of 100 beats per minute or higher.

The pulse oximeter 336 may be any type of pulse oximeter. For example, the pulse oximeter 336 may be a reflective pulse oximeter, a clip pulse oximeter or another type of pulse oximeter. The pulse oximeter 336 may operate to obtain data regarding the blood oxygenation levels, pulse patterns and/or heartrate information. The data obtained by the pulse oximeter 336 is sent to the computer module 334 for analysis.

The sensor 338 may be any type of biometric sensor, including but not limited to one or more of electrodes, a pulse oximeter, a glucose sensor, a respiratory sensor, a thermometer or another sensor device. The additional data obtained by the sensor 338 is sent to the computer module 334 for analysis. In some embodiments, the biometric sensing device 330 included additional sensors not shown herein.

The biometric sensing device 330 also includes the first electrode 340 and the second electrode 342. The two electrodes 340 and 342 operate to detect electrical signals within the patient's body that indicate heart activity. The computer module 334 receives readings from the electrodes 340 and 342 and analyzes the readings to determine a patient's heartrate.

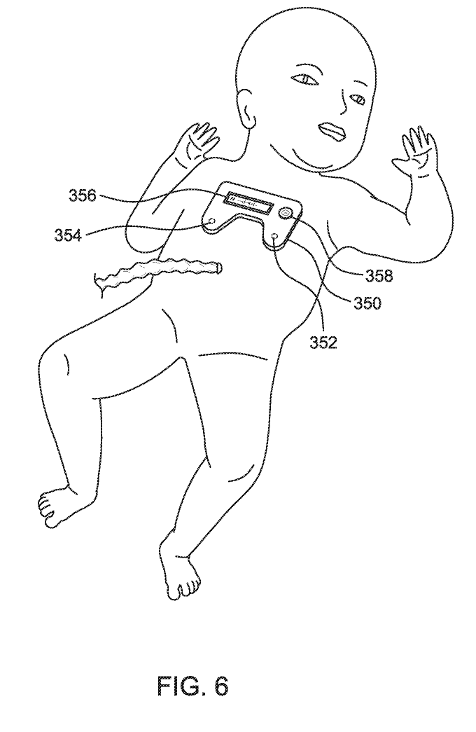

FIG. 6 shows another embodiment of a biometric patch 350 placed on the chest of a newborn. The patch 350 is similar to other embodiments of the patch discussed in conjunction with other figures herein. The patch 350 includes a first electrode 352 and a second electrode 354 (shown in dotted lines to indicate their placement on the bottom of the patch 350) as well as a display 356 and a button 358.

In this embodiment, the patch 350 has an alternative shape to the other embodiments shown. Specifically, the patch 350 includes two sections that extend separately away from the display 356. In this embodiment, the sections extend downward—away from the infant's head—and look similar to an upside down set of rabbit ears. The upside-down, rabbit ear shape of the patch 350 may allow for an improved view of the infant's abdomen while keeping the display 356 in focus for a medical professional. Other embodiments may have the sections directed upwards—toward the infant's head—or to one of the sides. One having ordinary skill in the art will recognize that the patches described herein may vary in shape, size, thickness, weight and other characteristics.

The first electrode 352 is located in one of the sections and the second electrode 354 is located in the other section. The location of the electrodes 352 and 354 in the extended sections may allow for better placement of the electrodes 352 and 354 to obtain accurate signals without interfering with a medical professional observation and work with the infant. In addition, placing the electrodes 352 and 354 away from the other electrical components in the patch 350 may reduce the likelihood of electrical interference. This may reduce or eliminate the application of insulation within the patch 350 for the purpose of preventing electrical interference.

The display 356 and button 358 may operate in the same way as other displays and controls discussed elsewhere herein. Specifically, the display 356 may provide relevant medical data to the medical professional working with the infant including the heartrate, oxygenation percentage, waveforms and/or other information. In addition, the display 356 may provide the information or indications for the information in a variety of formats. The button 358 may operate as an on/off control or a feature control for the patch 350.

FIG. 7 shows an embodiment of a pulse oximeter 400 that may be part of a biometric sensing system such as those discussed above or operate as a stand-alone device. The pulse oximeter 400 includes a band 402 to wrap around an infant's umbilical cord and a housing 404 attached to the band 402 by a wire connection 406.

In some embodiments, the housing 404 includes an umbilical cord clamp 408. In some embodiments, the housing 404 is designed to connect to the umbilical cord clamp 408. The housing 404 includes a computer module 412 and a display 410. The display 410 may be any type of visual output device, including a light array, a digital display, etc. In this embodiment, the display 410 shows an oxygenation percentage (93%) and a pulse waveform. In some embodiments, the housing 404 may operate as a visual output also. For example, the housing 404 may comprise a transparent or semi-transparent material and an internal LED or other light source that operates to light up the material allowing the housing 404 to light up.

The computer module 412 includes elements of a computer including but not limited to a processor, memory and input/output elements. A power source may be included in the computer module 412 or separately within the biometric sensing device housing 404. In some embodiments, the computer module 412 may include communication elements to receive and/or transmit signals with other external devices. The computer module 412 controls the operation of the pulse oximeter 400 through receiving data from biometric sensor components, evaluating the received data to determine biometric information and providing the applicable output to the display 410 for communication to an observer of the pulse oximeter 400. The actual output may be based upon standardized requirements stored in the memory of the computer module 412. For example, the computer module 412 may direct the display 410 to provide specific color outputs correlated to the calculated oxygenation percentage scaled from the time of birth of the patient, such as a red output for an oxygenation percentage under 67% at three minutes after birth, a yellow output for an oxygenation percentage between 67% and 72% at three minutes after birth and a green output for an oxygenation percentage above 72% at three minutes after birth.

The computer module 412 facilitates operation of the oxygen saturation sensor components including the light source 414 and the optical sensor 416. The light source 414, such as an LED, operates to provide light to the umbilical cord. The optical sensor 416 detects light that has passed through the umbilical cord. The computer module 412 calculates the oxygen saturation in the blood based upon the differences in the characteristics of the light emitted by the light source 414 and detected by the optical sensor 416. In some embodiments, the pulse oximeter 400 may include multiple light sources 414 and/or optical sensors 416. In addition, some embodiments may use light sources 414 that produce different light characteristics, such as a visible light source and an infrared light source.

The band 402 also includes attachment sections 418 and 420. These sections 418 and 420 may include corresponding attachment elements to allow the band to be secured around an infant's umbilical cord, including hook and loop tape, adhesives, friction materials and/or other attachment mechanisms or materials. In this embodiment, the attachment section 420 covers more of the band 402 than attachment section 418. This additional coverage by attachment section 420 allows for the band to vary in circumference and accommodate a variety of umbilical cord sizes.

Prior to attaching the pulse oximeter 400 to an infant, a medical professional may remove the pulse oximeter 400 from a sterile packaging, such as a TYVEK bag. In addition, the medical professional may turn on the pulse oximeter 400, select settings, start a clock associated with the infant's time of birth, etc. Following any set-up steps that may or may not be necessary depending on the embodiment of the pulse oximeter 400, the pulse oximeter 400 will be attached to the infant.

To attach the pulse oximeter 400, a medical professional will attach a clamp 408 to the infant's umbilical cord. In embodiments which include the clamp 408 with the housing 404, attaching the clamp 408 will also attach the housing 404. In other embodiments, the housing 404 may be attached to the clamp 408 after the clamp 408 is in place. The clamp 408 will be applied to the umbilical cord at a distance from the infant's abdomen that is at least sufficient to allow attachment of the band 402 on the umbilical cord between the clamp 408 and the infant's abdomen. For example, a medical professional may attach the clamp 408 four inches from the infants abdomen. One having ordinary skill in the art will recognize that the distance may vary along the umbilical cord depending on the medical professional's assessment of the needs for the infant as long as the circulation through the umbilical cord provides an accurate correlation to the oxygenation and pulse readings for the sensor elements.

Once the housing 404 is in place, the medical professional will wrap the band 402 around the umbilical cord between the clamp and the infant's abdomen and connect the attachment section 418 to the attachment section 420. When the attachment sections 418 and 420 are connected, the band 402 will be firmly wrapped around the umbilical cord without cutting off or inhibiting the circulation in the umbilical cord. In addition, the band 402 will hold the light source 414 and the optical sensor 416 in place for the pulse oximeter 400 to operate properly.

The band 402 may be placed in any location below the clamp 408 within the length of the connector 406. The connector 406 may be any length. In order to limit unnecessary material that may impede care or may get tangled, the connector 406 will be short. In some embodiments, the connector 406 will be less than three inches. In some embodiments, the connector 406 may be between half an inch and two inches. In some embodiments, a medical professional may use bandages, wraps, tapes and/or other materials to hold the connector 406 and/or the other portions of the pulse oximeter 400 in place and limit the likelihood of tangles.

Once the pulse oximeter 400 is attached to the infant, the pulse oximeter 400 will gather data from the light source 414 and/or optical sensor 416, any additional sensors and/or any external data sources in communication with the pulse oximeter 400. The computer module 412 will evaluate relevant data to determine biometric information about the baby, such as a heartrate and an oxygenation percentage. The computer module 412 will output relevant results, or indications thereof, on the display 410. In addition, the computer module 412 may transmit data and/or evaluation results to an external device.

Medical professionals can review the relevant data on the display 410 which is located on or near the abdomen of the infant on which the professional is attending. Providing the display 410 on the abdomen of the patient allows the medical professional to remain focused on the newborn while also monitoring the biometric data relevant to potential emergency situations.

In some embodiments, the display 410 and/or the housing 404 may provide a color output indicative of the heartrate or oxygenation of the patient relative to medical guidelines stored in the memory of the computer module 412. Providing the indicative color may allow the medical professional to recognize relevant information without modifying their focus from other medical activities for the newborn. For example, a medical professional may notice the display 410 and/or the housing 404 change from yellow to green indicating that the newborns oxygenation improved to a safer level.

In some embodiments of a biometric sensing system, other wirelessly networked components, such as other sensor devices, may provide additional data to the pulse oximeter 400. In some embodiments, the computer module 412 may output relevant data to the display 410 and/or the housing 404. In some embodiments, the computer module 412 may analyze the data to determine an output. During such an analysis, the computer module 412 may compare data or determined information to reference information stored in memory of the computer module 412 and provide an indication based upon the results of the comparison. Reference information may include medical guidelines, historical trends, prior patient or mother data points, custom references by the medical professional and/or other information.

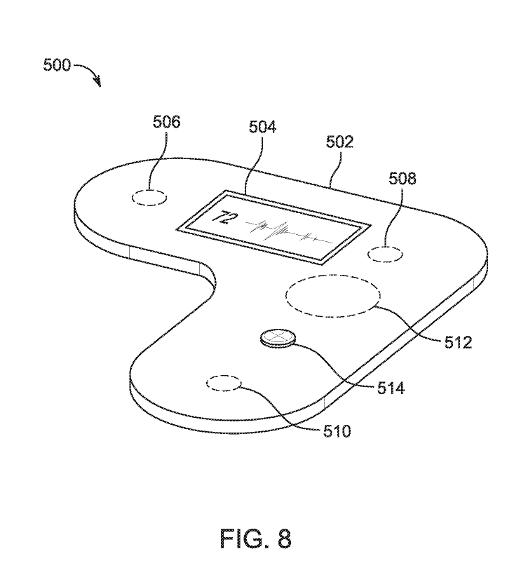

FIG. 8 shows another embodiment of a biometric sensing device 500 comprising a patch 502. The patch 502 is similar to other embodiments of the patch discussed in conjunction with other figures herein. The patch 502 includes a first electrode 506, a second electrode 508, a third electrode 510 and a sensor 512 (shown in dotted lines to indicate their placement on the bottom of the patch 502) as well as a display 504 and a button 514.

In this embodiment, the patch 502 has an alternative shape to the other embodiments shown. Specifically, the patch 502 creates an “L” shape with a section extending downward from the display 504 on one side of the patch 502. Other embodiments may have the extended section directed upwards or to one of the sides relative to the layout of the display 504. One having ordinary skill in the art will recognize that the patches described herein may vary in shape, size, thickness, weight and other characteristics.

This embodiment also illustrates the patch with three electrodes 506, 508 and 510. One skilled in the art will recognize that the number and placement of the electrodes may vary depending on the application and needs for the biometric sensing device 500. In an emergency situation, a design with fewer electrodes may be beneficial to avoid delays in attaching the device properly and time for the device to acquire and evaluate multiple data points. For example, acquiring an infant's heartrate provides sufficient information for an emergency resuscitation guidelines and a complete ECG screening with multiple specifically placed electrodes is unnecessary and introduces additional problems during the emergency situation.

In this embodiment, the first electrode 506 is located on one side of the display 504, the second electrode 508 is located on the opposite side of the display 504 and the third electrode 510 is located in the downwardly extended section. The location of the electrodes 506, 508 and 510 may allow for obtaining improved accuracy through additional signal interpretation based upon the data associated with each of the electrodes 506, 508 and 510.

The sensor 512 may be any type of biometric sensing element or elements, including one or more of a pulse oximeter, a glucose sensor, a respiratory sensor, a thermometer or another sensor device. The sensor 512 will obtain data regarding a patient's oxygenation, pulse patterns, glucose levels, respiration, temperature and/or other biometric data and send the data to a computer module (not shown) similar to those discussed elsewhere herein. The computer module may output the data to the display 504, analyze the data and provide a result to the display 504, store the data or calculated information and/or communicate the data to an external device.

The display 504 and button 514 may operate in the same way as other displays and controls discussed elsewhere herein. Specifically, the display 504 may provide relevant medical data to the medical professional working with the infant including the heartrate, oxygenation percentage, waveforms and/or other information. In addition, the display 504 may provide the information or indications for the information in a variety of formats. The button 514 may operate as an on/off control or a feature control for the patch 502.

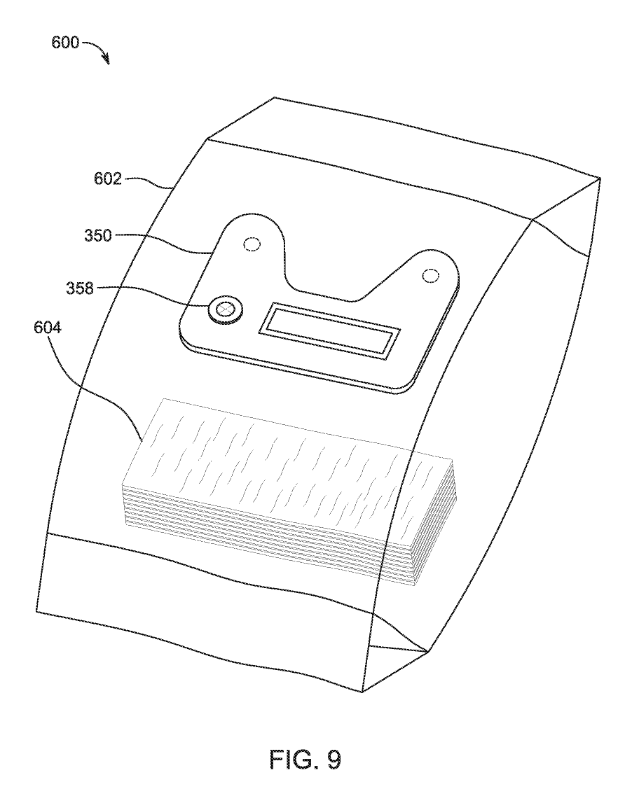

FIG. 9 shows an embodiment of a biometric sensing kit 600 including the biometric patch 350 described in further detail regarding FIG. 6. As discussed, the patch 350 is similar to other embodiments of the patch discussed in conjunction with other figures herein.

In this embodiment, the kit 600 includes a sealed package 602 containing the patch 350 and wipes 604. In some embodiments, the interior of the sealed package 602 provides a sterile environment to maintain sterility of the patch 350 and wipes 604. The sealed package 602 is shown as a bag in this embodiment, such as a TYVEK bag, which may be constructed from a variety of materials, such as a plastic or polyester material. Alternative, structures for the sealed package 602 may also be used including clam shell packages, vacuum sealed packages, boxes and other packaging options for medical components. In some embodiments, the sealed package 602 may be partitioned to hold the contents separately. The sealed package 602 may also include design elements to make opening the package safe and efficient, such as perforations, weakened tear lines, tear-away strips, a zip-top connection and/or other easy open options.

In this embodiment, the sealed package 602 includes one or more wipes 604. The wipes 604 may be used by a medical professional to clean the area of a patient for applying the patch 350. For example, the medical professional may use the wipe 604 to clean the chest of a newborn immediately prior to the application of the patch 350. For another example, a medic may wipe away dirt, blood and other material from a person's chest in the field prior to applying the patch 350. In some embodiments, the wipes 604 may be tailored for the specific use. For example, the wipes 604 for use on a newborn may contain a specified cleaning mixture designed to safely remove the vernix covering as well as other biological materials while also being soft to protect the newborn's delicate skin. In some embodiments, alternative cleaning components may be included instead of the wipes 604, such as cloths, sponges, a container of a cleaning liquid, and/or other cleaning materials.

During the preparation time prior to use of the patch 350, the medical professional may turn on the patch 350 by pressing the button 358 prior to removing the patch from the package. In addition, the button 358 may be used to select features for the patch 350 best suited for the anticipated medical use.

While patch 350 is shown in the kit 600, any of the patches described herein may be included in the kit 600. In addition, some kits 600 may include additional elements of a biometric sensing system. For example, an alternative kit 600 may include the patch 200 and the pulse oximeter 400 (each discussed above).

Embodiments of the kit 600 may be customized for specific applications. For example, a military medic's kit may include the patch 502, a fingertip pulse oximeter and a series of wipes 604 designed from a strong material and containing a disinfecting chemical.

The devices, systems and/or kits may be tailored for specific purposes based on intended use in a variety of situations, including patient transport activities, emergency medical transport activities, medical triage activities, military medical activities, remote healthcare activates, neonatal activities and other activities.

The invention being thus described and further described in the claims, it will be obvious that the same may be varied in many ways. Such variations are not to be regarded as a departure from the spirit and scope of the invention and all such modifications as would be obvious to one skilled in the art are intended to be included within the scope of the apparatuses, systems and methods described.

1. A biometric sensor patch comprising:
a patch body having a top layer and a bottom layer, wherein said patch body is configured to be suitable for application to a person's skin and wherein said top layer is configured to be facing away from said person when the patch is applied;
a sensor array in said bottom layer of said patch body comprising a first electrode and a second electrode configured to sense electrical activity in said person;
a display in said top layer of said patch body;
a power source within said patch body;
a computer module within said patch body that is operably connected to said sensor array, said display and said power source, wherein said computer module receives power from said power source, receives sensor data from said sensor array and provides an output to said display based on an analysis of said sensor data; and
wherein said display is configured to visually communicate said output to an observer.

2. The biometric sensor patch of claim 1 wherein said patch body is configured to be suitable for application to a newborn baby's chest.

3. The biometric sensor patch of claim 1 wherein said sensor array comprises a pulse oximeter.

4. The biometric sensor patch of claim 1 wherein said sensor array comprises at least one of a glucose sensor, a respiratory sensor and a thermometer.

5. The biometric sensor patch of claim 1 wherein said patch includes a communication module within said patch body.

6. The biometric sensor patch of claim 1 wherein said computer module comprises a processor that facilitates operation of said patch and analyzes said sensor data, and a memory that stores reference data for said analysis.

7. The biometric sensor patch of claim 6 wherein said analysis of said sensor data comprises said processor comparing said sensor data to said reference data stored in said memory.

8. The biometric sensor patch of claim 7 wherein said output is based upon said comparison of sensor data to said reference data.

9. The biometric sensor patch of claim 1 wherein said display visually communicates a heartrate based on said sensor data.

10. The biometric sensor patch of claim 1 wherein said display visually communicates a heartrate waveform based on said sensor data.

11. The biometric sensor patch of claim 1 wherein said sensor array comprises a third electrode configured to sense electrical activity in said person.

12. A biometric sensor system comprising:
a patch configured to be suitable for application to a person's skin comprising:
a top layer and a bottom layer, wherein said top layer is configured to be facing away from said person when the patch is applied,
a sensor array in said bottom layer of said patch comprising a first electrode and a second electrode configured to sense electrical activity in said person,
a display in said top layer of said patch that is configured to communicate
a visual output to an observer,
a power source within said patch,
a communication module within said patch configured to wirelessly communicate over a near-field communication standard,
a computer module within said patch that is operably connected to said sensor array, said display, said power source and said communication module, wherein said computer module receives power from said power source, receives sensor data from said sensor array, analyzes said sensor data and provides results for said visual output to said display, and wherein said computer module facilitates communication via said communication module;
an external device comprising
an external device communication module configured to wirelessly communicate over a near-field communication standard, and
an external device computer module configured to facilitate operation of said external device and communication via said external device communication module.

13. The biometric sensor system of claim 12 wherein said patch is configured to be suitable for application to a newborn baby's chest.

14. The biometric sensor system of claim 12 wherein said analysis determines a heartrate based on said sensor data.

15. The biometric sensor system of claim 12 wherein said sensor array comprises a pulse oximeter and said analysis determines a heartrate and an oxygenation percentage based on said sensor data.

16. The biometric sensor system of claim 12 wherein said external device is a sensor configured to obtain biometric data from said person, wherein said sensor transmits said biometric data via said external device communication module, and wherein said patch receives said biometric data via said communication module and said analysis comprises analyzing said sensor data and said biometric data.

17. The biometric sensor system of claim 16 wherein said sensor is at least one of a pulse oximeter, a glucose sensor, a respiratory sensor and a thermometer.

18. The biometric sensor system of claim 16 wherein said sensor is an umbilical cord pulse oximeter.

19. The biometric sensor system of claim 18 wherein said umbilical cord pulse oximeter includes a housing configured to attach to an umbilical cord clamp, wherein said housing includes an umbilical cord display.

20. The biometric sensor system of claim 12 wherein said external device is at least one of a monitor, a computer and a mobile communication device, wherein said external device receives biometric information via said external device communication module from said patch.

21. The biometric sensor system of claim 20 wherein said external device compares said biometric information to reference information stored in said external device and provides an output based on results from said comparison.

22. The biometric sensor system of claim 20 wherein said external device processes said biometric information to remove identifying information to create de-identified information, stores said de-identified information and aggregates said de-identified information with additional de-identified information from other sources.

23. A method for acquiring a newborn's biometric information for emergency care of the newborn, comprising the steps of:
providing a biometric patch configured to be suitable for application to the newborn's skin, wherein the biometric patch comprises:
a sensor array in a bottom layer of said patch comprising a first electrode and a second electrode configured to sense electrical activity in said newborn,
a display in a top layer of said patch that is configured to communicate a visual output to an observer, and
a computer module within said patch that is operably connected to said sensor array and said display;
applying the biometric patch to said newborn's chest;
observing said visual output on said display and wherein said visual output is based on an analysis by said computer module of sensor data acquired by said sensor array and is configured to indicate heartrate data.

24. The method of claim 23 wherein said visual output is a color output correlated to the heartrate data, wherein the color output is based on a medical guideline for newborn heartrates.

25. The method of claim 23 further comprising the step of pressing a button on said biometric patch when the newborn is born to establish a time of birth and begin a timer for elapsed time from the time of birth.

26. The method of claim 25 wherein said analysis is based on medical correlations of biometric data tied to elapsed time from the time of birth.
